(19) United States
(12) Patent Application Publication
(10) Pub. No.: US 2021/0366029 A1
(43) Pub. Date: Nov. 25, 2021

(54) Integrated Pharmaceutical Sales

(71) Applicant: MOBA Tech. LLC, Texarkana, TX (US)

(72) Inventors: Melannie Oller, Texarkana, TX (US); Brandy Alexander, Texarkana, TX (US)

(73) Assignee: MOBA Tech. LLC, Houston, TX (US)

(21) Appl. No.: 17/322,450

(22) Filed: May 17, 2021

Related U.S. Application Data
(60) Provisional application No. 63/028,675, filed on May 22, 2020.

(51) Int. Cl. G06Q 30/06; G16H 70/40; G16H 70/20; G16H 40/20; G16H 20/10; G06Q 30/00; G06Q 50/26; G06Q 50/20; G06Q 10/08; G06Q 30/02; G06F 8/60; G06F 3/0481; G06F 3/0482; G06T 11/00; G09B 5/08

(52) U.S. Cl. CPC G06Q 30/0641; G06Q 10/1095; G16H 70/20; G16H 40/20; G16H 20/10; G06Q 30/0633; G06Q 30/0625; G06Q 30/018; G06Q 50/26; G06Q 50/2057; G06Q 10/0833; G06Q 30/0277; G06F 8/60; G06F 3/04817; G06F 3/0482; G06T 11/00; G09B 5/08; G06Q 30/016; G16H 70/40

(57) ABSTRACT

Methods and systems for a software application for the marketing and sale of prescription drugs. Pharmaceutical companies must adapt to an ever changing, ever shrinking sales environment. Systems and methods described below may reach those targeted providers and provide every service that a human sales rep may provide. Discussed further below, the methods and systems provide video details, samples, co-pay cards, full product information, web page links, access to CE courses, Q and A links, printable insurance snapshot, product launch dates, virtual lunch and learn scheduling, partnered pharmacies, specialty pharmacies, convention links, printable and orderable detail literature and more.

### BACKGROUND

Over the past ten years, it has become increasingly difficult for a pharmaceutical company to market products with their manned salesforce. Many rules and regulations have been put in place to limit the number of Drug Reps into an office or ban them all together. Many offices tied to or owned by large hospital groups require access to be only through entities such as Vender Pro and others who charge over three hundred dollars per year per rep. In all, Pharmaceutical Companies and their salesforces are largely restricted from the access they once enjoyed.

This has created a real problem for the marketing of new branded drugs, and access is most certainly only going to continue to decrease with time. There are many factors causing the shutout such as increased time constraints on providers, increased EMR documentation time, increased number of patients seen, and current medical emergency facing the country.

### BRIEF DESCRIPTION OF THE DRAWINGS

These drawings illustrate certain aspects of some examples of the present disclosure and should not be used to limit or define the disclosure.

FIG. 1 is a communication diagram between a processing device and a network storage manager.

FIG. 2 highlights the interrelated components of a processing device.

FIG. 3 illustrates the architecture of between the user interface and communication.

FIG. 4 shows a graphical user interface provided by a processing device.

FIG. 5 shows a second graphical user interface provided by a processing device.

FIG. 6 shows results of actions performed within the second graphical user interface.

FIG. 7 is a workflow for prescription drug sale and marketing.

### DETAILED DESCRIPTION

This disclosure may generally relate to methods for marketing and selling of prescription drugs. Pharmaceutical companies must adapt to an ever changing, ever shrinking sales environment. Systems and methods described below may reach those targeted providers and provide every service that a human sales rep may provide. Discussed further below, the methods and systems provide video details, samples, co-pay cards, full product information, web page links, access to CE courses, Q and A links, printable insurance snapshot, product launch dates, virtual lunch and learn scheduling, partnered pharmacies, specialty pharmacies, convention links, printable and orderable detail literature and more. Additionally, the systems and methods may have zero barriers to cross, zero liability of reps caring illnesses from clinic to clinic. zero lock out, unlimited reach and frequency, zero off label discussions, respecting provider time, lessening patient irritation due to drug rep infringement on office staff and Provider and evolving the industry sales into what it must become in order to continue to be effective in an increasingly changing sales environment.

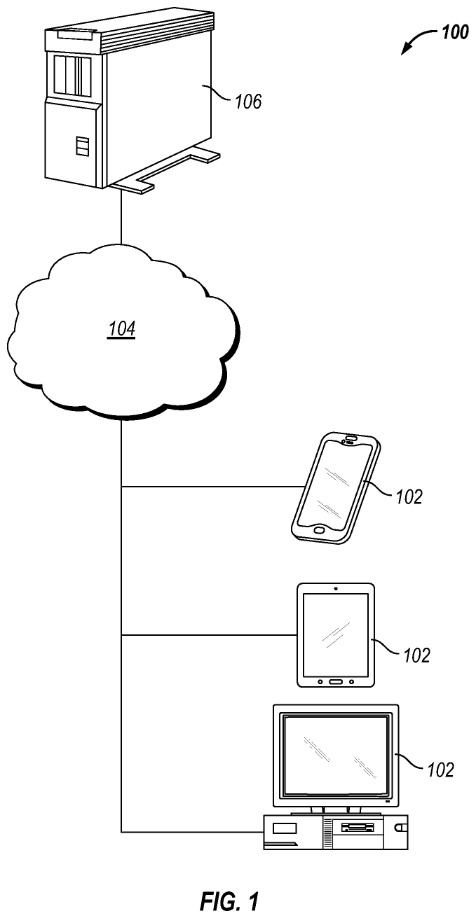

FIG. 1 illustrates an example of an environment suitable for practicing various embodiments described below. As illustrated, an integrated system 100 may operate and function to facilitate the sale, procurement of, and knowledge distribution of prescription drugs. Integrated system 100 may include one or more processing devices 102, a network 104, and network offsite storage 106. Network offsite storage 106 may be any suitable storage location for program code and related data for applications installed on processing devices 102. Network offsite storage 106 may serve as a host that preserves and provides access to software that has been installed on processing devices 102. Network offsite storage 106 may further operate and function to copy and maintain all files for an application, such as program code, data, and other documents, or may comprise a portion of the files, such as saved data or documents. Network offsite storage 106 may be implemented on a server using well known components of hardware or software. In some embodiments, information, data, and/or the like may be stored on network offsite storage 106 in any type of file format.

Network offsite storage 106 may employ various security features, such as secured logins, authentication, and encryption in order to protect its information. For example, network offsite storage 106 may restrict access of a processing device 102 to information corresponding to its device identifier. As another example, an entity may be allowed to access information for only those processing devices 102 to which it manages or to only those devices having software signed by that entity. Those skilled in the art will recognize that a variety of security policies and features may be employed to protect network offsite storage 106. During operations, network offsite storage 106 may maintain a list or database of applications that are authorized for each of processing device 102. For example, network offsite storage 106 may comprise respective lists or mappings indicating various aspects of the configuration of processing device 102, such as an identifier for processing device 102, applications installed, version information, devices that are related to processing device 102, and/or the like. Thus, any type of information that indicates the state and configuration of software or firmware installed on processing device 102 may be maintained in network offsite storage 106. Those skilled in the art will recognize that network offsite storage 106 may employ various lists, data structures, and databases to maintain this information. As illustrated, network offsite storage 106 may communicate with processing device 102 through network 104.

Network 104 provides a communication infrastructure between each processing device 102 and network offsite storage 106. Network 104 may be any type of network, such as a wide-area network, metropolitan-area network, or local-area network. In addition, network 104 may comprise both wired and wireless networks. In examples, network 104 may be implemented on the Internet, which is the well-known global network of interconnected computers, enabling users to share information. The components and protocols employed by network 104 are well known to those skilled in the art.

Processing devices 102 may be any computing device used by a user. Processing devices 102 may be mobile computing devices, such as mobile telephones, mobile smart-phones, or some other type of mobile device. Processing devices 102 may be configured to run an operating system that requires some or all of its software and code to have been securely installed. Thus, if software is delivered or installed in an unauthorized state to processing devices 102, the devices may be unable to fully execute the code instructions included in the software because they have not been properly installed. Additionally, there may be any number of processing devices 102 at any similar or different location accessing network offsite storage 106 through network 104.

Processing devices 102 may be any number of different types of computing devices, including desktop computers, laptop computers, handheld computers, personal digital assistant (PDA) devices, mobile telephone, media play device, and the like. For purposes of illustration, various embodiments related to a mobile device are provided. However, one skilled in the art will recognize that the embodiments disclosed may be applied to any type of computing device.

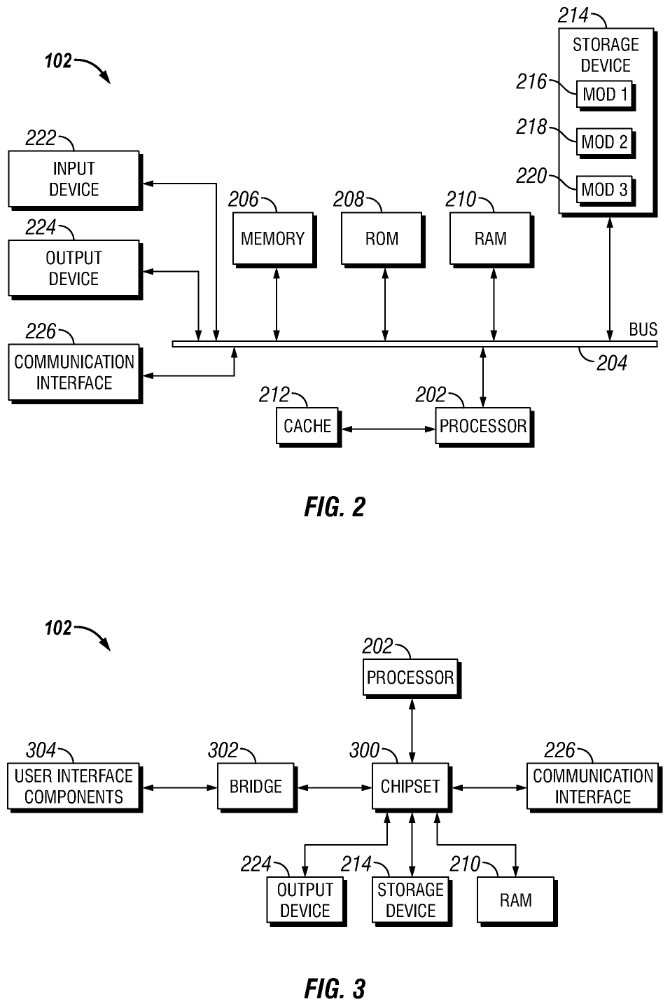

FIG. 2 illustrates an example processing device 102 which may be employed to perform various steps, methods, and techniques disclosed herein. Persons of ordinary skill in the art will readily appreciate that other system examples are possible. As illustrated, processing device 102 includes a processing unit (CPU or processor) 202 and a system bus 204 that couples various system components including system memory 206 such as read only memory (ROM) 208 and random-access memory (RAM) 210 to processor 202. Processors disclosed herein may all be forms of this processor 202. Processing device 102 may include a cache 212 of high-speed memory connected directly with, in close proximity to, or integrated as part of processor 202. Processing device 102 copies data from memory 206 and/or storage device 214 to cache 212 for quick access by processor 202. In this way, cache 212 provides a performance boost that avoids processor 202 delays while waiting for data. These and other modules may control or be configured to control processor 202 to perform various operations or actions. Other system memory 206 may be available for use as well. Memory 206 may include multiple different types of memory with different performance characteristics. It may be appreciated that the disclosure may operate on processing device 102 with more than one processor 202 or on a group or cluster of computing devices networked together to provide greater processing capability. Processor 202 may include any general-purpose processor and a hardware module or software module, such as first module 216, second module 218, and third module 220 stored in storage device 214, configured to control processor 202 as well as a special-purpose processor where software instructions are incorporated into processor 202. Processor 202 may be a self-contained computing system, containing multiple cores or processors, a bus, memory controller, cache, etc. A multi-core processor may be symmetric or asymmetric. Processor 202 may include multiple processors, such as a system having multiple, physically separate processors in different sockets, or a system having multiple processor cores on a single physical chip. Similarly, processor 202 may include multiple distributed processors located in multiple separate computing devices but working together such as via a communications network. Multiple processors or processor cores may share resources such as memory 206, cache 212, or may operate using independent resources. Processor 202 may include one or more state machines, an application specific integrated circuit (ASIC), or a programmable gate array (PGA) including a field PGA (FPGA).

Each individual component discussed above may be coupled to system bus 204, which may connect each and every individual component to each other. System bus 204 may be any of several types of bus structures including a memory bus or memory controller, a peripheral bus, and a local bus using any of a variety of bus architectures. A basic input/output (BIOS) stored in ROM 208 or the like, may provide the basic routine that helps to transfer information between elements within processing device 102, such as during start-up. Processing device 102 further includes storage devices or computer-readable storage media such as a hard disk drive, a magnetic disk drive, an optical disk drive, tape drive, solid-state drive, RAM drive, removable storage devices, a redundant array of inexpensive disks (RAID), hybrid storage, or the like. Storage device 214 may include software modules 216, 218, and 220 for controlling processor 202. Processing device 102 may include other hardware or software modules. Storage device 214 is connected to the system bus 204 by a drive interface. The drives and the associated computer-readable storage devices provide nonvolatile storage of computer-readable instructions, data structures, program modules and other data for processing device 102. In one aspect, a hardware module that performs a particular function includes the software component stored in a tangible computer-readable storage 214 in connection with the necessary hardware components, such as processor 202, system bus 204, and so forth, to carry out a particular function. In another aspect, the system may use a processor and computer-readable storage to store instructions which, when executed by the processor, cause the processor to perform operations, a method, or other specific actions. The basic components and appropriate variations may be modified depending on the type of device, such as whether processing device 102 is a small, handheld computing device, a desktop computer, or a computer server. When processor 202 executes instructions to perform “operations”, processor 202 may perform the operations directly and/or facilitate, direct, or cooperate with another device or component to perform the operations.

As illustrated, processing device 102 employs storage device 214, which may be a hard disk or other types of computer-readable storage devices which may store data that are accessible by a computer, such as magnetic cassettes, flash memory cards, digital versatile disks (DVDs), cartridges, random access memories (RAMs) 210, read only memory (ROM) 208, a cable containing a bit stream and the like, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with processing device 102, an input device 222 represents any number of input mechanisms, such as a microphone for speech, a touch-sensitive screen for gesture or graphical input, keyboard, mouse, motion input, speech and so forth. An output device 224 may also be one or more of a number of output mechanisms known to those of skill in the art. In some instances, multimodal systems enable a user to provide multiple types of input to communicate with processing device 102. Communications interface 226 generally governs and manages the user input and system output. There is no restriction on operating on any particular hardware arrangement and therefore the basic hardware depicted may easily be substituted for improved hardware or firmware arrangements as they are developed.

As illustrated, each individual component describe above is depicted and disclosed as individual functional blocks. The functions these blocks represent may be provided through the use of either shared or dedicated hardware, including, but not limited to, hardware capable of executing software and hardware, such as a processor 202, that is purpose-built to operate as an equivalent to software executing on a general-purpose processor. For example, the functions of one or more processors presented in FIG. 2 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 208 for storing software performing the operations described below, and random-access memory (RAM) 210 for storing results. Very large-scale integration (VLSI) hardware embodiments, as well as custom VLSI circuitry in combination with a general-purpose DSP circuit, may also be provided.

The logical operations of the various methods, described below, are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. Processing device 102 may practice all or part of the recited methods, may be a part of the recited systems, and/or may operate according to instructions in the recited tangible computer-readable storage devices. Such logical operations may be implemented as modules configured to control processor 202 to perform particular functions according to the programming of software modules 216, 218, and 220.

In examples, one or more parts of the example processing device 102, up to and including the entire processing device 102, may be virtualized. For example, a virtual processor may be a software object that executes according to a particular instruction set, even when a physical processor of the same type as the virtual processor is unavailable. A virtualization layer or a virtual “host” may enable virtualized components of one or more different computing devices or device types by translating virtualized operations to actual operations. Ultimately however, virtualized hardware of every type is implemented or executed by some underlying physical hardware. Thus, a virtualization compute layer may operate on top of a physical compute layer. The virtualization compute layer may include one or more virtual machines, an overlay network, a hypervisor, virtual switching, and any other virtualization application.

FIG. 3 illustrates an example processing device 102 having a chipset architecture that may be used in executing the described method and generating and displaying a graphical user interface (GUI). Processing device 102 is an example of computer hardware, software, and firmware that may be used to implement the disclosed technology. Processing device 102 may include a processor 202, representative of any number of physically and/or logically distinct resources capable of executing software, firmware, and hardware configured to perform identified computations. Processor 202 may communicate with a chipset 300 that may control input to and output from processor 102. In this example, chipset 200 outputs information to output device 124, such as a display, and may read and write information to storage device 114, which may include, for example, magnetic media, and solid-state media. Chipset 200 may also read data from and write data to RAM 110. A bridge 202 for interfacing with a variety of user interface components 204 may be provided for interfacing with chipset 200. Such user interface components 204 may include a keyboard, a microphone, touch detection and processing circuitry, a pointing device, such as a mouse, and so on. In general, inputs to processing device 102 may come from any of a variety of sources, machine generated and/or human generated.

Chipset 300 may also interface with one or more communication interfaces 226 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 202 analyzing data stored in storage device 214 or RAM 210. Further, processing device 102 receive inputs from a user via user interface components 304 and execute appropriate functions, such as browsing functions by interpreting these inputs using processor 202.

In examples, processing device 102 may also include tangible and/or non-transitory computer-readable storage devices for carrying or having computer-executable instructions or data structures stored thereon. Such tangible computer-readable storage devices may be any available device that may be accessed by a general purpose or special purpose computer, including the functional design of any special purpose processor as described above. By way of example, and not limitation, such tangible computer-readable devices may include RAM, ROM, EEPROM, CD-ROM or other optical disk storage, magnetic disk storage or other magnetic storage devices, or any other device which may be used to carry or store desired program code in the form of computer-executable instructions, data structures, or processor chip design. When information or instructions are provided via a network, or another communications connection (either hardwired, wireless, or combination thereof), to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general-purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

In additional examples, methods may be practiced in network computing environments with many types of computer system configurations, including personal computers, hand-held devices, multi-processor systems, microprocessor-based or programmable consumer electronics, network PCs, minicomputers, mainframe computers, and the like. Examples may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

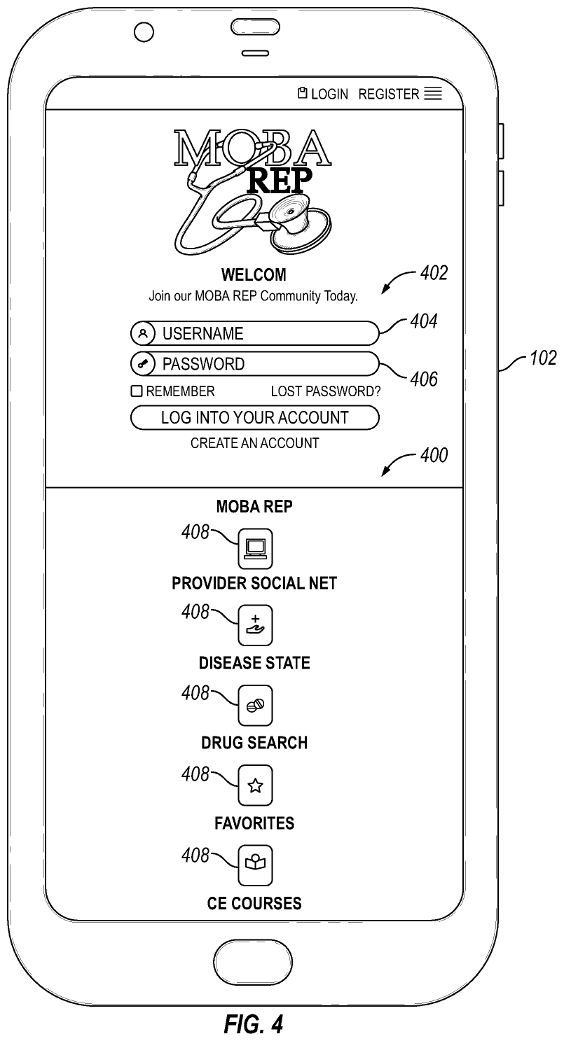

FIG. 4 illustrates communication interface 226 of processing device 102 (e.g., referring to FIG. 2) for a pharmaceutical application with the purpose of marketing and selling of prescription drugs. The pharmaceutical application may be downloaded and installed on processing device 102 from network offsite storage 106 utilizing network 104 (e.g., referring to FIG. 1). One of ordinary skill would know and understand the downloading and installing operations used to place the pharmaceutical application on processing device 102.

The pharmaceutical application may include a configurable graphical user interface (GUI) home overlay 400 (“home overlay 400”). Home overlay 400 may include a login section 402 for identifying a user of processing device 102. Login section 402 may include a username entry form 404 and a password entry form 406. Both sections may check the user against a database in network offsite storage 106 (e.g., referring to FIG. 1) to confirm identity and approved access to the application.

Further illustrated in FIG. 4 are a plurality of user interface (UI) buttons 408. UI buttons 408 have a set of system object attributes associated with each UI button 408. Each attribute determines whether a display object for the system object may be rendered in home overlay 400. This attribute may be set by the system automatically, or by a user input through certain programs or system functionalities. For example, selecting a UI button 408 may take a user to a feature of the application identified by the UI button 408. As illustrated, there is a UI button 408 for, but is not limited to, Provider Social Net, Disease State, Drug Search, Favorites, and/or CE Courses. Each feature may access data on network offsite storage 106. Specifically, each feature may access an individual database or multiple databases. This allows each feature to populate data and information for the user to review and select. It should be noted that there may be any number of UI buttons 408 for any number of features. In examples, home overlay 102 may be customized to include any number of UI buttons 408 for any number of features.

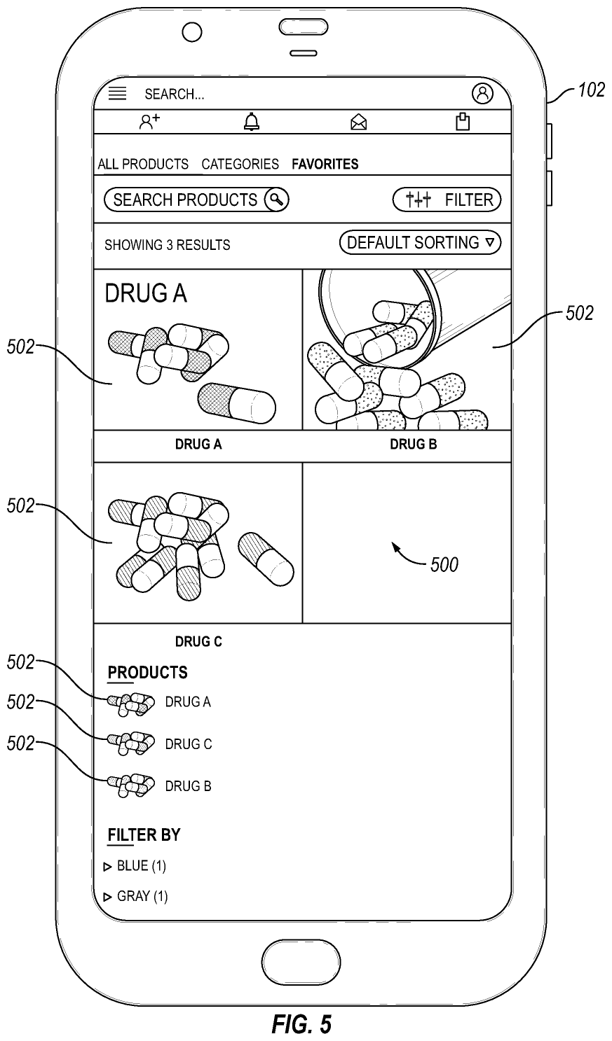

FIG. 5 illustrates a configurable GUI secondary overlay 500 (“overlay 500”) for the Drug Search feature. Processing device 102 may display overlay 500 that may be selected with the appropriate UI button 408 on home overlay 400 in FIG. 4. In the Drug Search feature, a user may search for a drug by product name, categories of drugs, and/or the like. Searching may populate one or more items 502 based on search criteria. Items 502 may be displayed in any suitable form for a user to select and may be filtered by the user using any suitable criteria specified by the user. Each item 502 may link the user to further information on the selected product.

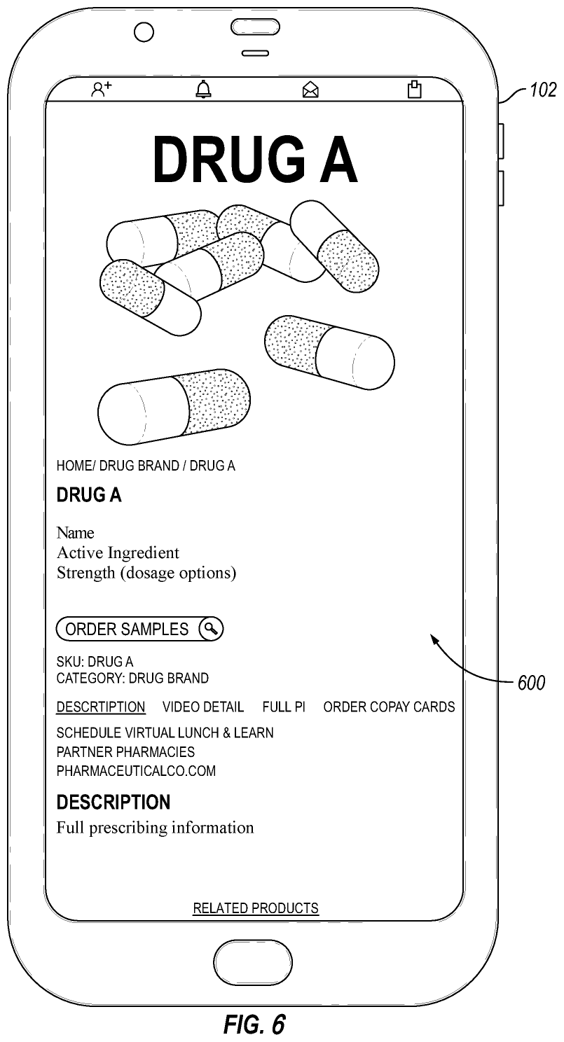

FIG. 6 illustrates a configurable GUI tertiary overlay 600 (“overly 600”) which further identifies a selected item 502 from FIG. 5. Processing device 102 will display overlay 600 which may include a picture of selected item 502 as well as a description of item 502. Additionally overlay 600, item 502 may be ordered for the user or to be shipped to a patient.

Referring back to FIG. 4, UI buttons 408 may access any feature available for the marketing and selling of prescription drugs. Additionally, each UI button 408 may access overlays similar to overlay 500 and overlay 600 (e.g., referring to FIGS. 5 and 6) that may be specific to the feature accessed by UI button 408.

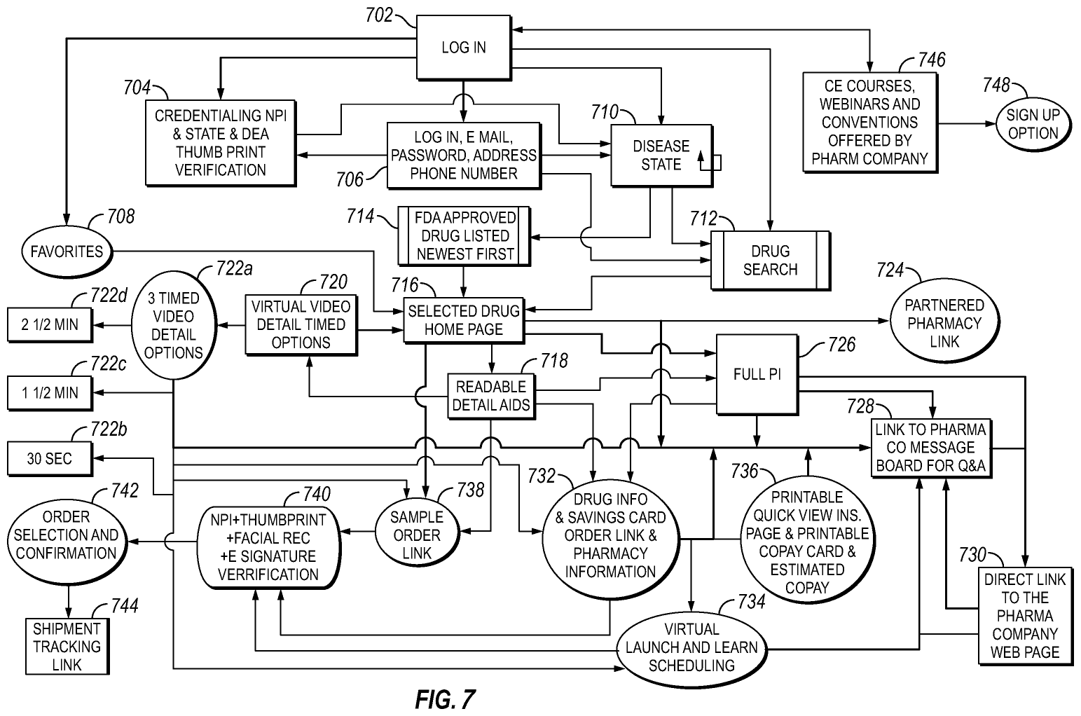

FIG. 7 illustrates a conceptual block diagram of an environment on processing device 102 that supports different features that operate and function on processing device 102. The conceptual block diagram is identified as workflow 700 for an integrated approach to the sale and marketing of prescription drugs. Workflow 700 may operate, run, and/or function on processing device 102 and is to be displayed on communication interface 226 (e.g., referring to FIG. 2) while utilizing the processing structures identified above. As a user inputs information possibly through input device 222, processing device 102 (e.g., referring to FIG. 2) navigates through workflow 700 and populates communication interface 226 with different GUI overlays comprising information directed by the specific location within workflow 700. Workflow 700 illustrates the structural working between each feature that is accessible by UI buttons 408 (e.g., referring to FIG. 4). During implementation, workflow 700 may call processing device 102 (e.g., referring to FIG. 2) to access network offsite storage 106 through network 104. Workflow 700 may begin by accessing the software application in block 702 by logging into the application. Logging into the application may require a user to provide a password, biometric scan, face identification, any combination thereof and/or the like from a user input. By logging into the software in block 702 the user may be able to accesses the base features. Base features may include Credentialing in block 704, settings in block 706, a disease state in block 708, and/or a drug search in block 710.

Block 704 depicts one of the UI buttons 408 (e.g., referring to FIG. 4)—“CE Courses” for obtaining credentials. The processing device 102 (e.g., referring to FIG. 2) may display UI button 408 linked to “CE Courses” in block 704 on any overlay disclosed above. This may be performed by communication interface 226 and may prompt for a user input possibly through input device 222 to apply for and gain credentials for National Provider Identifier (NPI), various State government regulations, and Drug Enforcement Agency (DEA). Each credential method may be tailored and updated to reflect current laws, regulations, and/or requirements for any agency or government entity. One option that may be available for a user is thumbprint verification. In examples, a user may scan a thumb print of the user using the software. This may be done on a mobile platform or stationary device. The scanned thumbprint may then be sent to a selected agency or government entity for verification and storage.

In block 706, standard setting operations may be available to custom tailor the software for the user. For example, login passwords may be changed, email communication may be programmed, contact information such as phone numbers and addresses may be added, and/or the like. Additionally, setting in block 706 may adjust how the user may view and interact with the software. Additionally, in block 708, favorites may be stored. For example, favorites may include custom setting, selected prescription drugs, and/or other software inputs or outputs the user may utilize on numerous occasions. Favorites may be ordered by disease state or by drugs used most frequently by the user. Typically, a provider will store drugs most often used in their practice by drug name in their favorites for quick access to sample ordering, literature and copay-card ordering, or full product information reference for patient counseling.

Block 710 depicts one of the UI buttons 408 (e.g., referring to FIG. 4)—“Disease State” which may be selected by a user input possibly through input device 222. Upon selection of a disease state, processing device 102 (e.g., referring to FIG. 2) may populate communication interface 226 with on any overlay discussed above. Information displayed may provide, but is not limited to, a disease, illness, or condition in which a provider is treating in a patient. Additionally, a disease state may also include a disease in which a provider wants to see the newest FDA approved drugs for their personal growth and education enabling them to be up to date on the best possible options available for the patients they treat. Once the disease state is selected, the data populates with the newest FDA approved Pharmaceutical Prescription drugs on the market provided by the contracted Pharmaceutical companies. The data is populated by date and may be scrolled through and selected by the provider for further details, videos, and all services provided.

Block 712 depicts one of the UI buttons 408 (e.g., referring to FIG. 4)—“Drug Search”. As previously stated, a drug search is performed based on the identified diseases selected in overlay 500. During this search, any number of prescription drugs, over the counter drugs, and/or the like that may be used to treat the disease may be populated for review in overlay 500. In block 714, selected drugs may be compared to a Food and Drug Administration (FDA) approved prescription drug list. In examples, the populated drugs, FDA information, and website may be populated for reviewed by the user in block 716 as previously described in overlay 600. This populated list may be displayed in such a way as to allow the user to determine which drug or prescription drug may be chosen. By selecting a drug or prescription drug, a pop-up screen or new information may be display for the user to review. For example, in block 718, additional details about the drug or prescription drug may be displayed in any overlay discussed above. Such information may include, but is not limited to, side effects, reactions with other drugs, whom the drug may be prescribed to, what the drug may treat, and/or like. In block 720, additional video of the selected drug details may be found and run in any overlay discussed above. For example, in blocks 722a-d videos for advertisements, how to use the drug, and/or other helpful information may be run on a video for the user to watch.

Selection of a drug or prescription drug may open up a partnered specialty pharmacy link in block 724. Here, if a specialty Pharmacy is utilized, that information will be available for reference or print in overlay 600. Allowing the provider to locate pharmacies with chosen product on the shelf as well as instructions on how to send the prescription electronically to the pharmacy. Also available, is the possibility of a contract pharmacy liaison which automatically searches out lowest price pharmacy for products offered. Additionally, in block 726, a full prescribing information (PI) yielding UI button 408 (e.g., referring to FIG. 4)—“Provider Social Net”. Block 726 may be populating any overlay discussed above with the chosen drug or prescription drug. The information populated for block 726 may allow for providers to connect across all specialties and practices. Block 726 will give providers a platform which they may announce new products and educate targeted clients about their products. Clients may expand on the platforms they access and learn about the companies providing to them. Additionally, providers may give customers information current regulations do not allow. For example, a link to one or more pharmaceutical company message boards for questions and answers in block 728. In block 730 a direct link to the pharmaceutical company web page may be populated in any overlay discussed above and displayed on communication device 226 of processing device 102 (e.g., referring to FIG. 2) for the user to select possibly through input device 222. This may direct the user to the pharmaceutical company for further information. Similarly, in block 732, drug information and previous order information may be displayed to the user. Previous order information may include the previous pharmacy the drug or prescription drug was ordered from. Further, payment information such as credit card information may be stored for future use.

During operations, the user may select a help button that may launch from block 732 and lead to block 734. In block 734, the help button may launch a program for scheduling virtual or in person meetings with drug representatives, pharmacy representative, and/or the like. Additionally, from block 732, a link to for payment options may be displayed. Selecting this link may populate insurance information, copay information, estimated cost, and/or the like in block 736.

Referring back to block 716, the displayed information may have a link for ordering the drug or prescription drug in overlay 600. This link will update the display to a sample order link in block 738. A verification requirement is performed in block 740, which may allow a user to order the selected drug or prescription drug. The verification step in block 740 may require a thumbprint scan, facial recognition, E-signature verification, and/or any combination thereof. After verification, in block 742, an order selection is place and confirmation is sent to the user to notify the user that the order was received. In block 744, the user may receiver tracking shipment updates from the postal carrier.

Additionally, one of the UI buttons 408 (e.g., referring to FIG. 4)—“Favorites” may be provided in any overlay discussed above. A user may tag any collection of information displayed in workflow 700 to a bookmarks function. Additionally, the most visited sites throughout a user's navigation through workflow 700 may be recorded and tagged in the bookmarks function. Every source tagged throughout workflow 700 within the bookmarks function may be displayed in any overlay discussed above.

Workflow 700 may also include an advertisement section in block 746 which may be displayed in any overlay discussed above. In block 746, continued education (CE) courses, webinars, and convention for pharmaceutical companies may be displayed to the user. This information may be displayed to the user in any suitable location on any suitable location discussed above. The user may select the specific advertisements, which may lead to sign-up options in block 748. The sign-up options may also be connected to ordering in block 738-744.

The improvement upon current technology is evident with the use of UI buttons 408 (e.g., referring to FIG. 4) and the other applications incorporated by workflow 700 help exhibit valuable information to customers in an evolvingly restrictive market. A targeted client base may now be expanded as pharmaceutical companies are provided a platform to release information and circumnavigate traditional human sales rep. Thus, a pharmaceutical company can provide specific and transparent information.

Additionally, the above-described systems and methods are an improvement over current technologies because the current technologies focus is to get the physicians in contact with a pharmaceutical representative whereas the systems and methods described above take the place of the pharmaceutical representative in its entirety. Systems and methods further provide a private professional social network putting providers in touch with their peers to discuss their experience with listed pharmaceutical drugs. Improvements over current technology also include offering video detail for each drug as well as listing partnered and specialty pharmacies. Still further, the methods and systems provide printable versions of all information as well as coupon and co-pay cards for immediate use in the office. MOBA also improves upon other technologies by offering accessibility to webinars and continuing education courses. Consequently, safer and more affordable products will achieve more commercial success than unhealthy and more expensive products.

The methods/systems/compositions/tools may include any of the various features disclosed herein, including one or more of the following statements.

Statement 1: A method may comprise accessing a pharmaceutical application stored at an offsite storage with a processing device, downloading the pharmaceutical application to the processing device, accessing one or more databases on the offsite storage through the pharmaceutical application, selecting a database with a user interface (UI) button on a home overlay, populating a second overlay with data from the database, and selecting one or more items populating on the second overlay from the database.

Statement 2. The method of statement 1, wherein the UI button accesses a Disease State feature.

Statement 3. The method of statement 2, wherein the Disease State feature accesses the one or more databases to search for one or more treatments used to treat a disease, an illness, or a condition.

Statement 4. The method of statement 3, wherein a third overlay displays one or more drugs that treat the disease, the illness, or the condition.

Statement 5. The method of statement 4, further comprising selecting the one or more drugs which populates the third overlay with information on the one or more dugs that were selected.

Statement 6. The method of statement 5, further comprising ordering one or more drugs that were selected.

Statement 7. The method of statements 1 or 2, wherein the UI button accesses a Drug Search feature.

Statement 8. The method of statement 7, further comprising populating one or more drugs based at least in part on a search entered by a user.

Statement 9. The method of statement 8, further comprising selecting the one or more drugs which populates a third overlay with information on the one or more dugs that were selected.

Statement 10. The method of statement 9, further comprising ordering one or more drugs that were selected.

Statement 11. The method of statements 1, 2, or 7, wherein the UI button accesses a Credentialing feature.

Statement 12. The method of statement 11, wherein the Credentialing feature accesses the database in the offsite storage that includes one or more licenses, thumbprint verification, National Provider Identifier, and Drug Enforcement Agency credentials for distribution of drugs.

Statement 13. The method of statements 1, 2, 7, or 11, wherein the UI button accesses Continued Education (CE) feature.

Statement 14. The method of statement 13, wherein the CE feature accesses the database in the offsite storage that populates data on one or more webinars or one or more conventions.

Statement 15. The method of statements 1, 2, 7, 11, or 13, further comprising logging into the offsite storage using a login section on the home overlay.

Statement 16. The method of statements 1, 2, 7, 11, 13, or 15, further comprising ordering the one or more items on the second overlay from a pharmacy through the pharmaceutical application.

Statement 17. The method of statement 16, further comprising tracking a shipment of the one or more items through the pharmaceutical application.

Statement 18. The method of statements 1, 2, 7, 11, 13, 15, or 16, further comprising advertising one or more drugs on the home overlay.

Statement 19. The method of statements 1, 2, 7, 11, 13, 15, 16, or 18, further comprising accessing a pharmaceutical company from the home overlay.

Statement 20. The method of statements 1, 2, 7, 11, 13, 15, 16, 18, or 19, further comprising accessing one or more message boards from the home overlay.

The foregoing figures and discussion are not intended to include all features of the present techniques to accommodate a buyer or seller, or to describe the system, nor is such figures and discussion limiting but exemplary and in the spirit of the present techniques.

What is claimed is:

1. A method may comprise:
accessing a pharmaceutical application stored at an offsite storage with a processing device;
downloading the pharmaceutical application to the processing device;
accessing one or more databases on the offsite storage through the pharmaceutical application;
selecting a database with a user interface (UI) button on a home overlay;
populating a second overlay with data from the database; and
selecting one or more items populating on the second overlay from the database.

2. The method of claim 1, wherein the UI button accesses a Disease State feature.

3. The method of claim 2, wherein the Disease State feature accesses the one or more databases to search for one or more treatments used to treat a disease, an illness, or a condition.

4. The method of claim 3, wherein a third overlay displays one or more drugs that treat the disease, the illness, or the condition.

5. The method of claim 4, further comprising selecting the one or more drugs which populates the third overlay with information on the one or more dugs that were selected.

6. The method of claim 5, further comprising ordering one or more drugs that were selected.

7. The method of claim 1, wherein the UI button accesses a Drug Search feature.

8. The method of claim 7, further comprising populating one or more drugs based at least in part on a search entered by a user.

9. The method of claim 8, further comprising selecting the one or more drugs which populates a third overlay with information on the one or more dugs that were selected.

10. The method of claim 9, further comprising ordering one or more drugs that were selected.

11. The method of claim 1, wherein the UI button accesses a Credentialing feature.

12. The method of claim 11, wherein the Credentialing feature accesses the database in the offsite storage that includes one or more licenses, thumbprint verification, National Provider Identifier, and Drug Enforcement Agency credentials for distribution of drugs.

13. The method of claim 1, wherein the UI button accesses Continued Education (CE) feature.

14. The method of claim 13, wherein the CE feature accesses the database in the offsite storage that populates data on one or more webinars or one or more conventions.

15. The method of claim 1, further comprising logging into the offsite storage using a login section on the home overlay.

16. The method of claim 1, further comprising ordering the one or more items on the second overlay from a pharmacy through the pharmaceutical application.

17. The method of claim 16, further comprising tracking a shipment of the one or more items through the pharmaceutical application.

18. The method of claim 1, further comprising advertising one or more drugs on the home overlay.

19. The method of claim 1, further comprising accessing a pharmaceutical company from the home overlay.

20. The method of claim 1, further comprising accessing one or more message boards from the home overlay.
